(19) United States
(12) Patent Application Publication
(10) Pub. No.: US 2003/0044432 A1
(43) Pub. Date: Mar. 6, 2003

(54) Acne treating composition

(76) Inventors: Vincent E. Manetta, Bordentown, NJ (US); Linda M. Mahoney, Phoenixville, PA (US); Michael T. Nordsiek, Berwyn, PA (US); Kodumudi S. Balaji, Lansdale, PA (US); Jay Dorrell, Vicksburg, MI (US)

Correspondence Address: H Eric Fischer, Synnestvedt & Lechner, 2600 Aramark Tower, 1101 Market Street, Philadelphia, PA, 19107-2950, US

(21) Appl. No.: 10/019,822

(22) PCT Filed: Jun. 28, 2000
(86) PCT No.: PCT/US00/18033

(30) Foreign Application Priority Data
Sep. 29, 2000 (GB) 0023891.5

(51) Int. Cl. A61K 31/192; A61K 9/00

(52) U.S. Cl. 424/400; 514/568

(57) ABSTRACT

A package comprising components which, upon being mixed, are capable of forming a pharmaceutical composition that is effective in treating acne, the composition tending to degrade prematurely, one of the components comprising an oxidizing agent and another of the components comprising an antibiotic which is effective against acne-associated bacterial species, the components separated one from the other in the package, one component having a viscosity within about 50% of the viscosity of the other component. In addition, a package comprising: (A) components which, upon being mixed, are capable of forming a pharmaceutical composition that is effective in treating acne, one of the components comprising a benzoyl peroxide gel and another of the components comprising a gel of erythromycin and hydroxypropylcellulose; and (B) containers for holding the components in the package separated one from the other; the components having viscosities such that, upon the application of a uniform forces to the components, substantially equal volumes of the components are capable of being dispensed simultaneously from the containers.

### FIELD OF THE INVENTION

The present invention relates to a pharmaceutical composition that tends to lose its effectiveness prematurely and to improved means for the effective use of such a composition. More particularly, the present invention relates to the packaging of constituents comprising such a composition and to an improved form of such composition, including, for example, a composition for topical application, such as a topical composition for the treatment of acne.

Acne is a common inflammatory disease which is very common at puberty and may continue for many years. It occurs in facial skin areas where sebaceous glands are the largest, most numerous, and most active. In its milder forms, acne is a superficial disorder which is evidenced by slight, spotty irritations and which can be treated satisfactorily by ordinary skin hygiene. However, in the more inflammatory types of acne, bacterial invasion of or about the pilosebaceous follicles occurs and results in the formation of pustules, infected cysts, and, in extreme cases, canalizing inflamed and infected sacs appear. These lesions may become extensive and leave permanent, disfiguring scars.

Therapeutic methods for treating acne are designed to prevent formation of new lesions and facilitate the healing of old lesions. Treatments include the systemic and topical administration of anti-acne agents such as antibiotics or synthetic Vitamin A analogs. In most cases, systemic treatment of acne is not desirable because of the risks of adverse side effects that are experienced by the user. For this reason, topical acne treatment compositions have been preferred.

The present invention relates to topical anti-acne compositions and packages which contain the components of the composition.

### REPORTED DEVELOPMENTS

Topical anti-acne compositions include, for example, one or more of the following: sulfur, resorcinol, salicylic acid, benzoyl peroxide, steroids and antibiotics. Exemplary antibiotics are disclosed in U.S. Pat. No. 3,969,516 (lincomycin family); BR Publication No. 1,594,314 (erythromycin); and U.S. Pat. No. 3,952,099 (tetracycline). Preparations containing a peroxide are reported in U.S. Pat. Nos. 3,535,422; 4,056,611; 4,387,107, and British Publication No. 1,594,314 and U.S. Pat. No. 4,497,794. Antibiotic-containing compositions which also include anti-inflammatory steroids are disclosed in U.S. Pat. No. 4,132,781.

Attempts to improve the effectiveness of topical antibiotic compositions for use in the treatment of acne have taken a number of approaches. One approach is reported in U.S. Pat. Nos. 3,989,815; 3,989,816, 3,991,203; 4,122,170; 4,316,893; 4,444,762; and EP 27,286, which disclose skin-penetrating vehicle compositions that reportedly increase the transdermal absorption of any physiologically active substance, including antibiotics. However, some penetrating agents are not compatible with antibiotics used to treat acne.

A further approach relates to the use of a composition which utilizes two different active components, such as erythromycin, clindamycin, Vitamin A acid or benzoyl peroxide. Compositions including mixtures of a peroxide and erythromycin are reported in British Publication No. 1,594,314. Acne-treating compositions comprising benzoyl peroxide, erythromycin, and hydroxyethylcellulose have also been disclosed in “The Formulation and Stability of Erythromycin-Benzoyl Peroxide in a Topical Gel, Vermeulen et al., International Journal of Pharmaceutics, 178, 137-141 (1999)”. U.S. Pat. No. 5,894,019 to Hess et al. discloses a topically applied pharmaceutical composition that is prepared by mixing a liquid solution of benzoyl peroxide (or other pharmaceutically active ingredient) that is dissolved in a liquid lipid with a hydrous gel that may also contain a pharmaceutically active ingredient, such as erythromycin. The gelling agent used to form the hydrous gel may include hydroxypropylcellulose. U.S. Pat. No. 5,466,446 to Stiefel et al, U.S. Pat. No. 5,733,886 to Baroody et al., and U.S. Pat. No. 5,767,098 to Klein et al. disclose acne-treating compositions which contain clindamycin and benzoyl peroxide.

The above compositions comprising a peroxide, such as benzoyl peroxide, and an antibiotic, such as erythromycin or clindamycin, may have stability limitations. When the peroxide and antibiotic are combined, the peroxide can partially or completely oxidize the antibiotic, rendering it ineffective. If the peroxide and antibiotic are provided separately, it is usually necessary to have a pharmacist combine the component containing the peroxide with the component containing the antibiotic to ensure that the composition is prepared properly, has the correct final concentrations of the peroxide and antibiotic, and to maximize the shelf life of the composition by postponing the mixing of the components until the time of sale. Another problem with these compositions is that once mixed, the oxidation reactions of the peroxide not only degrade the antibiotic, but also result in the accumulation of undesirable degradative products in the composition. An additional problem is that unused portions of certain acne-treating composition must be refrigerated to minimize degradation until use.

The present invention relates to improved means for using effectively anti-acne compositions that tend to lose their effectiveness prematurely and to improved forms of anti-acne compositions.

### SUMMARY OF THE INVENTION

In accordance with the present invention, there is provided a package comprising components which, upon being mixed, are capable of forming a pharmaceutical composition that is effective in treating acne, the composition tending to degrade prematurely, one of the components comprising an oxidizing agent and another of the components comprising an antibiotic which is effective against acne-associated bacterial species, the components separated one from the other in the package, one component having a viscosity within about 50% of the viscosity of the other component.

In preferred embodiments, the oxidizing agent is benzoyl peroxide, the antibiotic is selected from the group consisting of erythromycin and clindamycin, and the package is a dual packet laminated foil package.

Another aspect of the present invention provides a package comprising: (A) components which, upon being mixed, are capable of forming a pharmaceutical composition that is effective in treating acne, one of the components comprising a benzoyl peroxide gel and another of the components comprising a gel of erythromycin and hydroxypropylcellulose; and (B) containers for holding the components in the package separated one from the other; the components having viscosities such that, upon the application of a uniform force to the components, substantially equal volumes of the components are capable of being dispensed simultaneously from the containers.

Yet another aspect of the present invention is a composition for the treatment of acne comprising: (A) benzoyl peroxide; (B) erythromycin; and (C) hydroxypropylcellulose. Examples of preferred compositions may further comprise an additional gelling agent, an agent which inhibits degradative interactions between benzoyl peroxide and erythromycin, a base which increases the pH of the composition to a value at which said composition has an acceptable viscosity, an alcohol solvent for erythromycin, and a diluent.

Yet another aspect of the present invention provides a composition for the treatment of acne comprising: (a) benzoyl peroxide; (b) erythromycin; (c) hydroxypropylcellulose; (d) a gelling agent; (e) a surface active agent; (f) a base; (g) an alcohol; and (h) water. An example of a preferred gelling agent includes a hydroxylated vinylic polymer.

An example of a preferred base is sodium hydroxide. An example of a preferred alcohol used in the composition is specially denatured ethanol. An example of a surface active agent is dioctyl sodium sulfosuccinate.

The invention includes also within its scope the provision of a package containing a first acne-treating composition and a second acne-treating composition, the compositions tending to degrade chemically upon being mixed with one another, the package comprising a first chamber containing the first composition and a second chamber containing the second composition, each of the chambers having a sealed dispensing orifice, the orifices positioned adjacent to one another.

Another aspect of the present invention provides a package containing a first acne-treating composition and a second acne-treating composition, the compositions tending to degrade chemically upon being mixed with one another, the package comprising a first and second packet, the packets being attached to each other and each of the packets having a sealed dispensing orifice, the sealed dispensing orifices being positioned adjacent to each other so that, as the compositions are dispensed through the orifices in unsealed form, they are combined with each other.

An additional aspect of the present invention provides a package containing a first acne-treating composition and a second acne-treating composition, the compositions tending to degrade chemically upon being mixed with one another, the package comprising a first and second packet, each of the packets having a top which includes a dispensing orifice that has a tear-off tab seal, a bottom, two sides in a parallel relationship to one another, and an exterior face and an interior face, the first packet containing the first composition and the second packet containing the second composition, the interior faces of said packets adjacent to and facing one another such that the tear-off tabs can be simultaneously torn off such that the compositions are dispensed and combined with each other as they exit the packets through the dispensing orifices.

### DESCRIPTION OF THE DRAWINGS

FIG. 1 is a plan view of a dual-packet package.

FIG. 2 is a plan view of the package of FIG. 1 folded about axis A-A.

FIG. 3 is a cross-sectional view of a trilaminate package.

FIG. 4 is an exploded view of a pump for dispensing the compositions of the present invention.

FIG. 5 is a partial sectional view of the assembled pump depicted in FIG. 4.

### DETAILED DESCRIPTION OF THE INVENTION

The acne-treating composition of the present invention comprises a plurality of components which when combined tend to degrade prematurely. Degradation is considered premature when the components are mixed together and at least one of the components degrades at a rate faster than the rate at which it degrades (if at all) in the absence of the other component. As an example, in acne-treating compositions which contain an oxidizing agent and an antibiotic, the oxidizing agent will oxidize and thereby degrade the antibiotic at a much faster rate than the antibiotic would degrade in the absence of the oxidizing agent.

Components that degrade prematurely when combined may be used in the practice of the present invention and may be identified using techniques known.in the art. Examples of such components include, but are not limited to, oxidizing agents, gelling agents, and antibiotics.

The acne-treating composition of the present invention is provided in a package which -maintains the components separated from each other until the components are removed in whole or in part from the package. In preferred form, the package is designed to enable the user to extract readily from the package amounts of components which form an effective acne-treating composition.

Use of such a package allows long-term storage of the components at room temperature without degradation. The package can be carried conveniently by the patient because the contents do not require refrigeration. This increases patient compliance with administration of the composition. In addition, because the composition is mixed at the time of application, degradation is minimized. Furthermore, the proportions of active ingredients in the composition can be optimized by exact filling of packets and by the ease with which the patient can empty the packets simultaneously such that the final composition has the desired ratios of components.

The package of the present invention may be made from any suitable material, for example, paper, plastic, or metal foil, in laminated or composite form. The material functions to protect the components from exposure to air, moisture, light and other environmental factors which could change the physical and/or chemical nature of the components and to keep the components from contact with each other.

In preferred form, the package of the present invention includes chambers which maintain the components of the composition in separated form and which accommodate amounts of the components such that, when the components are removed from their chambers and brought together, they form a single effective acne-treating dose of the composition. For example, in preferred embodiments, the package contains a sufficient amount of the composition to apply a thin layer of the composition to the entire surface of the face (with the exception of the eyes and mouth) and sufficient amounts of the pharmaceutically active ingredients to treat effectively any acne present in the areas to which the composition is applied.

It is believed that the present invention will be used widely in the packaging of an acne-treating composition comprising a component which includes an oxidizing agent and a component which includes an antibiotic which is effective in treating an acne condition. As mentioned above, the oxidizing agent in such a composition tends to oxidize and thereby degrade the antibiotic when the two components are brought together. The present invention may also be used in acne-treating compositions which include retinoic acid.

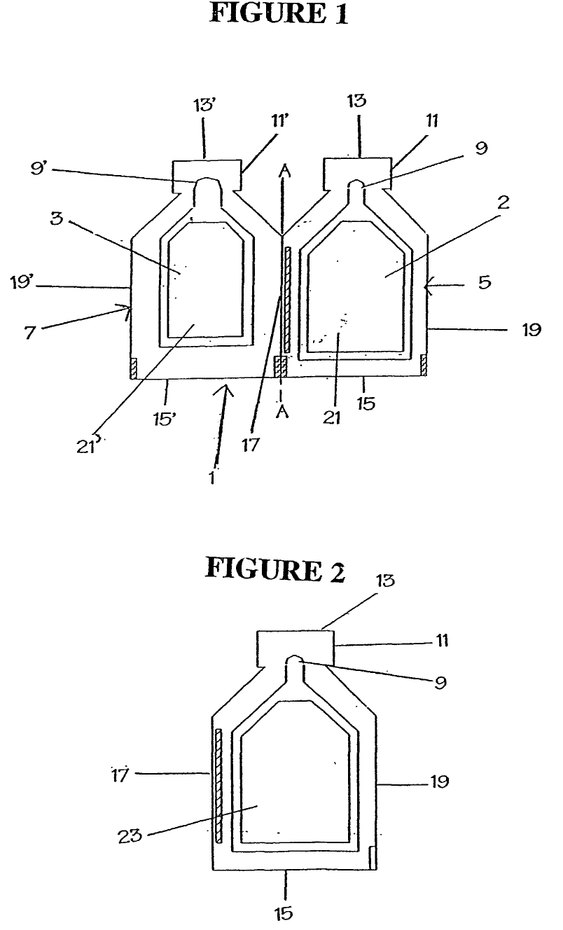

A particularly preferred embodiment of the package of the present invention is shown in FIG. 1, in which there is illustrated a dual-packet, laminated metal foil package 1 comprising a first packet 5 which includes a first chamber 2 containing a first component of the pharmaceutical composition and a second packet 7 which includes a second chamber 3 containing a second component of the composition. Each of the chambers 2 and 3 has a sealed dispensing orifice 9 and 9′ with tear-off tabs 11 and 11′. Each packet has a top portion 13 and 13′, a bottom portion 15 and 15′, a commonly shared side 17 and a second side 19 and 19′ in parallel relationship to each other, and an interior face 21 and 21′ and an exterior face (not shown in FIG. 1) in opposed relationship to each other. The first chamber 2 and second chamber 3 may be of equal or different size.

FIG. 2 shows the package of FIG. 1 in a form in which the packets have been folded about axis A and the interior faces 21 and 21′ of the packet are in apposition to one another and the exterior faces 23 and 23′ (not shown) face outwardly. The tear-off tabs 11 and 11′ of the packets are superimposed upon one another. This permits them to be removed simultaneously. After removal of the tabs, the components in chambers 2 and 3 can be dispensed and combined with each other as they exit packets 5 and 7 through the dispensing orifices 9 and 9′.

In preferred embodiments, the packages are comprised of two packets made of a multi-layered laminated foil. Preferably the laminate is a five-layer design consisting of three main barrier layers and two adhesive layers which lie between the barrier layers and hold the layers together. In preferred embodiments, the packets have a first innermost layer comprised of low density polyethylene. This layer is in contact with the composition. The second layer is an adhesive layer comprised of ethylene-acrylic acid copolymer which binds the first layer to the third layer. The third layer is a barrier layer made of aluminum foil. The fourth layer is comprised of ethylene-acrylic acid copolymer which serves as an adhesive layer binding the third layer to the fifth layer. The fifth and final layer is comprised of polyester which provides durability and tear- and puncture-resistance to the package. A preferred laminate for use in the present invention is available from Reynolds Metals Co.

For exemplary purposes, the use of the package of FIGS. 1 and 2 is described in connection with an acne-treating composition comprising an oxidizing agent and an antibiotic. Accordingly, one of the chambers of the package contains the oxidizing agent and the other chamber contains the antibiotic. To extract the oxidizing agent and antibiotic from the package, both packets thereof are opened simultaneously by tearing off their respective tear-off tabs. The packets are then squeezed by the user until the contents of the chambers are emptied substantially. The contents typically exit their respective orifices in the form of a ribbon. The oxidizing agent and the antibiotic component can be mixed conveniently to form the acne-treating composition by the user's dispensing the contents into the palm of one hand and using the fingers of the other hand to mix the components together by use of a swirling motion. The composition is then applied to the site or sites in need of treatment.

The package of FIGS. 1 and 2 can be characterized as a single-dose package which contains a sufficient amount of each component to form a volume of composition which can be applied as a thin layer to the entire surface of the face (with the exception of the eyes and mouth) with a minimal amount of the composition left over. Any remaining composition may be discarded. Because of the relatively small amounts of components in the package, the package can be considered also to be a “single-use” package which is thrown away after its contents are emptied.

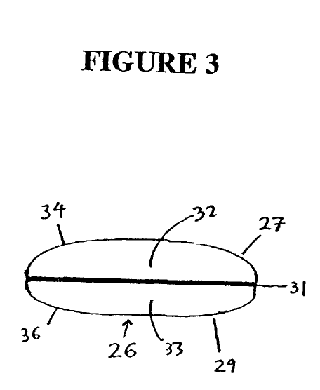

FIG. 3 illustrates a cross-sectional view of another package embodiment in which there is illustrated a “trilaminate” package 26 comprising a first packet 27 and a second packet 29, having a common internal barrier layer 31, preferably a single layer of aluminum foil or other suitable material known in the art. This internal barrier layer 31 is in contact with the first 32 and second 33 compositions within the first 27 and second 29 packets. The internal barrier layer 31 is sealed along its perimeter to a first exterior barrier layer 34, and a second exterior barrier layer 36. Both the first 34 and second 36 exterior barrier layers are preferably comprised of a single layer, of aluminum foil or other suitable material known in the art.

A “multiple use” package is capable of holding relatively large amounts of components for use in forming the treating composition. The multiple-use package is similar to the single-use package in that the components of the acne-treating composition are kept separate from one another and mixed when the product is dispensed. In preferred embodiments, the multiple-use package may include a dose-metering pump which dispenses an equal amount of both components of the acne-treatment composition.

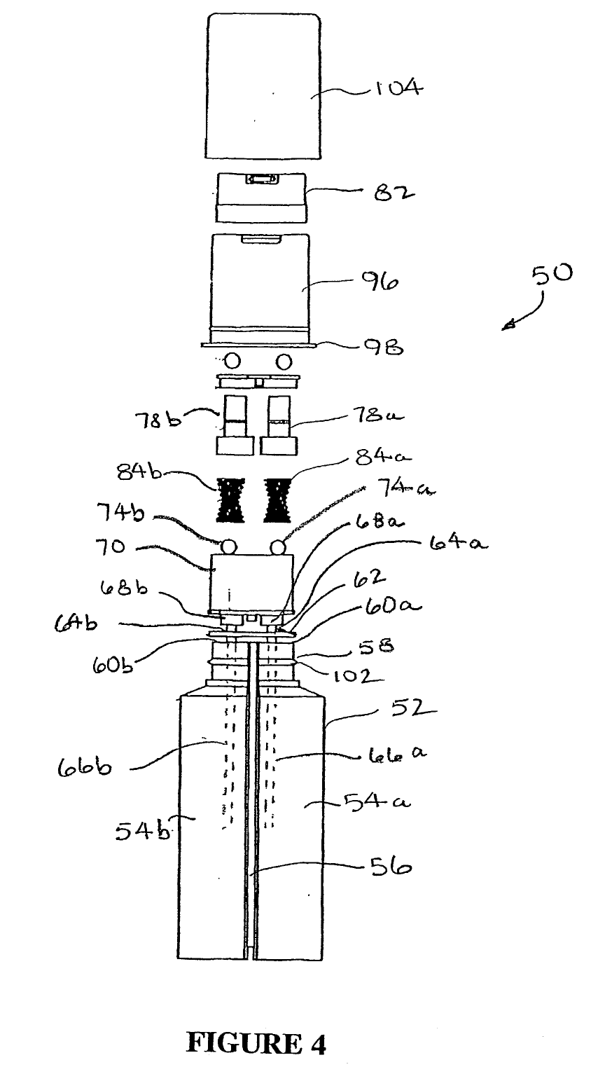

FIG. 4 shows an exploded side view of a pump 50 for dispensing a multi-component fluid formulation, for example, a topical anti-acne composition. The preferred pump embodiment shown is designed to accommodate a two-component formulation, although it is understood that the design is not limited to use with formulations having. only two components.

Pump 50 is suitable for use with lotions, creams, oils, gels and liquids and is designed to keep the components of the formulation separate while they are stored in the pump. Mixing of the components occurs only after they have been dispensed from the pump immediately before use. This feature is useful to extend the effective life of formulations, such as the anti-acne composition, which tend to degrade once the components are combined. The pump is also designed to dispense the components of the formulation in the proper relative amounts which provide maximum effectiveness of the formulation.

As seen in FIG. 4, pump 50 comprises a bottle 52 having two reservoirs 54a and 54b separated from one another by a bulkhead 56. The reservoirs hold the different components of the formulation and are attached to a common neck 58 which provides for attachment of the other pump components, described below, to the bottle 52. Neck 58 surrounds and defines two openings 60a and 60b in reservoirs 54a and 54b respectively, the openings permitting the components of the formulation to be loaded into the reservoirs. A liner 62 engages neck 58 and seals openings 60a and 60b to prevent premature mixing of the components within the pump.

Liner 62 has two through openings 64a and 64b through which dip tubes 66a and 66b extend into reservoirs 54a and 54b respectively. Dip tubes 66a and 66b provide conduits for drawing the formulation components from the reservoirs 54a and 54b to be dispensed by the pump. Each dip tube is connected to a respective valve seat 68a or 68b, the valve seats being attached to a receptacle 70 which sits atop neck 58.

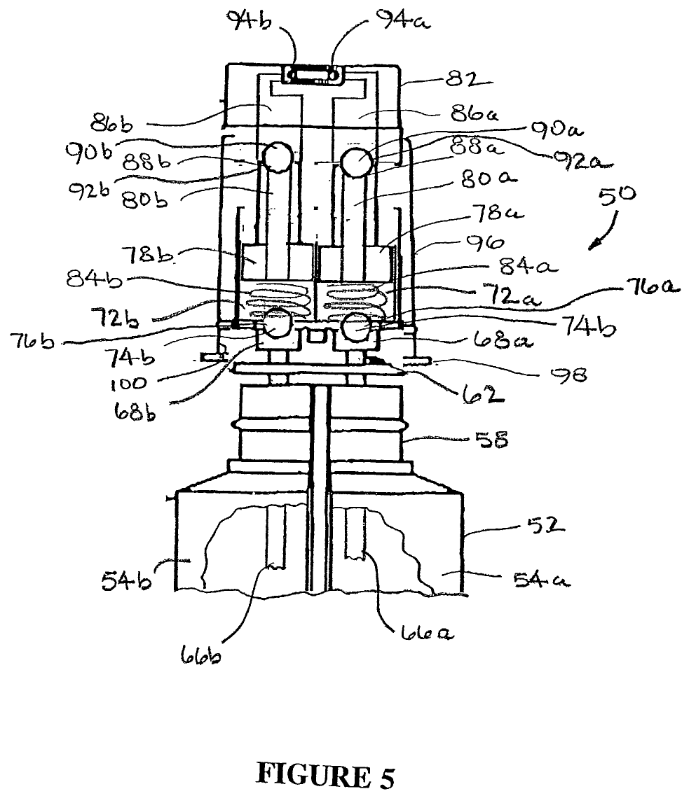

As best seen in FIG. 5, receptacle 70 has two cylinders 72a and 72b which are in fluid communication with dip tubes 66a and 66b through valve seats 68a and 68b respectively. Housed in each cylinder is a respective ball 74a or 74b. Balls 74a and 74b are preferably stainless steel and are sized to seat in respective valve seats 68a and 68b to form a pair of check valves 76a and 76b which allow components of the formulation to flow in one direction only, from the respective reservoirs 54a and 54b through the dip tubes 66a and 66b and into cylinders 72a and 72b as described in detail below.

Cylinders 72a and 72b each receive a respective piston 78a and 78b. The pistons are sized to sealingly interfit slidably within their respective cylinders and provide suction and pumping action when they are reciprocated within the cylinders during pump actuation. Pistons 78a and 78b have respective piston bores 80a and 80b which provide fluid communication between their respective cylinders 72a and 72b and a spout 82 attached to the tops of the pistons. Pistons 78a and 78b are each biased to a position away from check valves 76a and 76b by respective springs 84a and 84b housed within cylinders 72a and 72b between the valve seats 68a and 68b and the pistons 78a and 78b as shown.

As seen in FIG. 5, spout 82 has ducts 86a and 86b which are in fluid communication with piston bores 80a and 80b of pistons 78a and 78b respectively. A second set of valve seats 88a and. 88b are provided at the tops of piston bores 80a and 80b. A second set of balls 90a and 90b are positioned within ducts 86a and 86b respectively, the balls being sized to seat on respective valve seats 88a and 88b and form a second set of check valves 92a and 92b. Preferably, balls 90a and 90b are made of stainless steel. Ducts 86a and 86b are in fluid communication with respective dispensing nozzles 94a and 94b from which the components held in reservoirs 54a and 54b are dispensed to be mixed outside of pump 50.

As seen in FIGS. 4 and 5, most of the pump components including the spout 82; pistons 78a and 78b, check valves 76a, 76b, 92a and 92b, springs 84a and 84b, and receptacle 70 are housed within a retainer 96. Retainer 96 has a lower portion 98 adapted to engage neck 58 and retain the pump components to the bottle 52. Attachment of the retainer is preferably by an interference fit between the retainer inner surface 100 and raised ribs 102 surrounding neck 58. An over cap 104 fits over the retainer 96 and protects the moving parts of the pump against unintentional pump actuation.

Pump 50 is preferably made from various types of resilient, flexible plastic materials, such as polyethylene and polypropylene to cite examples. Plastics are advantageous because they are inexpensive, easily molded, substantially unbreakable, and chemically inert and, thus, will not affect the components of the formulation. Parts of the pump, such as the springs for biasing the pistons and the balls for the check valves, for which plastic is not suited are preferably made of stainless steel. Stainless steel provides a robust, durable material which also is substantially chemically inert and, thus, suitable for pump parts which will come into contact with the components of the formulation during pump operation.

Operation of the dual dispensing pump 50 is described with reference to FIG. 5. In operation, the user removes the over cap 104 exposing spout 82. With the pump 50 in an upright position, the user manually depresses spout 82. This causes the spout and attached pistons 78a and 78b to move downwardly with respect to the retainer 96, check valves 76a and 76b, liner 62, and bottle 52.

Pistons 78a and 78b move within their respective cylinders 72a and 72b compressing the formulation components within the cylinders as well as springs 84a and 84b. Balls 74a and 74b are firmly seated in valve seats 68a and 68b, operating as check valves to prevent the formulation components in the cylinders from being forced into respective reservoirs 54a and 54b. The formulation components are forced upwardly through the piston bores 80a and 80b in each piston respectively, the hydraulic pressure within each cylinder unseating balls 90a and 90b from valve seats 88a and 88b at the tops of the piston bores 80a and 80b. The formulation components are forced from the cylinders through the piston bores into ducts 86a and 86b in the spout, the components being dispensed from dispensing nozzles 94a and 94b in the ducts and into the hand of the user who mixes the components to form the complete formulation, ready for use.

Upon release of spout 82, springs 84a and 84b push the pistons 78a, 78b to their biased position spaced away from check valves 76a and 76b. Upward motion of the pistons creates a suction within the cylinders 72a and 72b and the piston bores 80a and 80b. The suction seats balls 90a and 90b on valve seats 88a and 88b to prevent formulation components in ducts 86a and 86b from being drawn back into the piston bores 80a and 80b. Simultaneously, the suction causes balls 74a and 74b to lift off their respective seats 68a and 68b as the formulation components are drawn up through dip tubes 66a and 66b from reservoirs 54a and 54b and into cylinders 72a and 72b. The pump is then ready to dispense the formulation components on the next actuation.

As explained above, each component of the formulation is pumped from its reservoir by its own piston-cylinder combination. The amount of a particular component dispensed from the pump is proportional to the volume of the cylinder swept by its piston, which pumps that particular component. Since the strokes of both pistons are equal, the relative amount of component dispensed from one reservoir in relation to the other reservoir is determined by the ratio of the cylinder diameters. Cylinders having equal diameters will have equal swept volumes and, thus, dispense equal amounts of formulation component from each reservoir. If the diameter of one of the cylinders is made smaller relative to the other cylinder, then the piston associated with the smaller cylinder will sweep a smaller volume, and a smaller amount of component will be dispensed from the respective reservoir. Thus, the pump can be designed to automatically dispense formulation components in the proper relative proportions for maximum effectiveness simply by having cylinders with different diameters, the ratio of the diameters being proportional to the desired ratio of formulation components to be dispensed.

The package embodiments of the present invention are designed to be used by the lay person who has an acne condition or a lay person who applies the composition to the individual with the condition. Accordingly, the packages are designed so that the user is able to dispense on a consistent basis effective amounts of the active ingredients comprising the pharmaceutical composition. Thus, the need for a pharmacist to prepare the composition is eliminated. The specific amount of each of the active ingredients present in the package will depend on the identity of the active ingredient, taking into account its known effect on treatment of the acne condition and the proportion of the ingredients that are desired in the composition that is formed by mixing the active ingredients. Also, the package may contain an optimal dosage of the active ingredients of the composition by including in each packet the appropriate volume and concentration of the active ingredient. For example, in the use of a peroxide/antibiotic composition, it may be desired that the peroxide comprises a greater proportion of the composition than the antibiotic.

It will be appreciated that the packets of the single-dose package, as described herein, contain relatively small volumes of the components from which the composition is formulated. For example, an embodiment of such a packet contains about 0.425 g of one component and about 0.425 g of a second component. The dispensed components are within plus or minus 0.025 g of the 0.425 g in the packet, allowing for about a 6% tolerance. The filling of the packets with effective dosage amounts of the components is essential to providing an effective acne-treating composition. This is difficult to accomplish with components that are in a form of the type described herein, for example, in gel form. In order to fill the packets with the accurate dosages of components, it has been found that it is important to rid the components of air trapped within the components. To avoid trapped air, the components are formulated preferably using a process which avoids the introduction of air into the components and removes trapped air from the components. This is accomplished by preparing the components under a vacuum, for example, about −15 to about −30 psi. By substantially eliminating trapped air from the components, it is possible to fill the packets or other appropriate containers with a high degree of accuracy. This, in turn, allows the lay person to prepare the acne-treating composition within the desired concentration parameters.

The form of the composition that is applied to the skin should be such that it adheres to the skin for a sufficient period of time to allow the active ingredients to act on the affected area. As an example, for embodiments which include a peroxide and an antibiotic, the composition will be in a form which adheres to the skin for a sufficient period of time for the peroxide to inactivate extracellular lipases and inhibit the formation of free fatty acids in the skin and for the antibiotic to reduce the concentration of acne-associated bacteria in the affected area. For this purpose, the acne-treating composition should have a viscosity such that the composition adheres to the skin. This requires that, when the components that make up the composition are combined together, they form a composition which has a viscosity such that the composition adheres to the skin. The forms of the components and the viscosities thereof can be different and vary substantially, provided that they can be combined to form a composition having the desired viscosity characteristics. For example, the components may be in forms ranging from liquids to solids. In preferred embodiments, as described in detail below, the component is in a form which has properties intermediate the liquid and solid states. Examples of such forms include gels, lotions, creams, salves, and ointments. In a particularly preferred embodiment, the components are in gel form.

The package of the present invention, as exemplified in FIGS. 1 and 2, includes the separated components in dosages which are effective in forming, upon mixing, an effective acne-treating composition. In preferred form, the package is designed and the components are formulated to enable the user to dispense on a consistent basis substantially all of the contents of the package. Accordingly, the contents are made available for combining into an acne-treating composition in which the components are present in the desired dosage. The preferred means of accomplishing this is to formulate components which have viscosities that enable the user to dispense the components from the package without undue effort. It is preferred also that the components be in the same form. For example, if one component is in gel form, it is preferred that the other component be in gel form. In addition, it is preferred that the components have similar viscosities. The term “similar viscosity” means that when a uniform “dispensing” force is applied to the separated components, substantially equal amounts of the components are dispensed simultaneously from their respective chambers. In a particularly preferred form, the components have viscosities, as measured by 1V-101 Brookfield Helipath T-F @ 1.5 rpm that are within about 50%, preferably within about 25% of each other.

Standard procedures known in the art may be used to determine viscosity. To perform the viscosity test, a test sample was transferred to a 1 lb jar (89 mm in diameter and 92 mm in height) while tapping on the jar to eliminate air. After the transfer, the jar was left to sit for one hour at 25.0° C. A Brookfield Model LV series rotational viscometer equipped with a helipath and a T-F spindle was used to measure the viscosity of the sample. The spindle of the viscometer was placed in the sample and the height of the spindle was adjusted so that the crosspiece of the spindle was covered by about ¼″ of the sample. The rotational speed of the spindle was set to 1.5 rpm. The helipath and the viscometer were simultaneously started. After one minute, the needle lock was engaged. When the needle was displayed in the window, the viscometer was shut off. A reading was taken and recorded on the 0-100 scale. Additional tests may be performed to obtain additional readings. If additional tests are performed, the viscometer rotation should be resumed and at least one rotation allowed prior to the release of the needle lock. Timing of the additional sample readings is to be commenced from the time of the release of the needle lock.

In particularly preferred embodiments, each of the components is a gel which has a viscosity of about 200,000 cps to about 500,000 cps.

There follows a description of exemplary ingredients comprising the acne-treating composition of the present invention, including a description of exemplary pharmaceutically active ingredients and exemplary ingredients that are combined therewith to place the composition in a suitable form for application to the face and for dispensing the components of the composition from the package of the present invention.

In the discussion which follows, the amount of an ingredient in the acne-treating composition is expressed in weight percent based on the total weight of the composition (% w/w-composition). The amount of an ingredient. in one of the components is expressed in weight percent based on the total weight of the component (% w/w-component).

The acne-treating composition includes an antibiotic which is effective against acne-associated bacterial species. An antibiotic is effective against acne-associated bacterial species if it kills or inhibits the growth of a bacterial population. The term “acne-associated bacterial species” refers to bacterial species found at the site of an acne lesion, in particular Propionibacterium acnes (P. acnes).

Exemplary antibiotics include lincomycin, clindamycin (a lincomycin derivative), and erythromycin and their pharmaceutically acceptable salts and esters such as their hydrochlorides and phosphates.

Lincomycin is a derivative of the amino acid trans-L-4-α-propyl-hydrinic acid coupled to a derivative of an octose substituted by a methylmercaptyl group. Lincomycin antibiotics are described in U.S. Pat. Nos. 3,475,407; 3,509,127; 3,544,551 and 3,513,155.

Clindamycin is the 7-deoxy,7-chloro derivative of lincomycin, and is otherwise known as methyl 7-chloro-6,7,8,trideoxy-6-[[(1-methyl-4-propyl-2-pyrrolidinyl)carbonyl]amino]-1-thio-L-threo-α-D-galacto-octopyranoside. In the use of clindamycin as the antibiotic, it is recommended that there be used clindamycin phosphate which is converted in vivo to active clindamycin.

A preferred antibiotic for use in the present invention is erythromycin, which is a macrolide antibiotic produced from a strain of Saccharopolyspora erythraea (formerly Streptomyces erythreus). It is a base and readily forms salts with acids. Erythromycin is otherwise known as [(3R*, 4S*, 5S*, 6R*, 7R*, 9R*, 11R*, 12R*, 13S*, 14R*)-4-[(2,6-dideoxy-3-C-methyl-3-O-methyl-α-L-ribo-hexopyranosyl)-oxy]-14-ethyl-7,12,13-trihydroxy-3,5,7,9,11,13-hexamethyl-6-[[3,4,6-trideoxy-3-(dimethylamino)-β-D-xylo-hexopyranosyl]oxy]oxacyclotetradecane-2,10-dione].

The antibiotic is present in the composition in a pharmaceutically effective concentration. Such concentration will vary depending on the antibiotic used and the nature of the individual being treated. For guideline purposes, it is recommended that the antibiotic be present at a concentration of about 0.5 to about 6% w/w-composition, preferably about 0.8 to about 5% w/w-composition. In particularly preferred embodiments of the invention utilizing erythromycin, the erythromycin is at a concentration of about 1.8 to about 4.5% w/w-composition. In preferred embodiments utilizing clindamycin, the clindamycin is at a concentration of about 0.7 to about 2% w/w-composition.

In addition to the antibiotic, the package of the present invention can include any other active ingredient that is effective in treating acne and that may be combined with an antibiotic to form a composition which tends to degrade prematurely. Examples of such active ingredients include corticosteroids, tretinoin, antifungal compounds, alpha-hydroxy acids and beta-hydroxy acids.

In preferred embodiments, the acne-treating composition preferably includes an antibiotic as described above and an oxidizing agent which inhibits the formation of free fatty acids in the skin primarily through inactivation of extracellular lipase enzymes. Preferably, the composition includes a “peroxide” oxidizing agent. The term “peroxide” means an organic compound containing an oxygen-oxygen bond capable of cleaving and forming oxygen free-radicals. A variety of peroxides may be used in the practice of the present invention. The peroxides include peroxyacids of carboxylic acids, peroxyesters of carboxylic acids and the dimeric product of carboxylic peroxyacids. Exemplary peroxides include t-butyl peroxyesters of straight and branched chain aliphatic carboxylic acids, and dimeric peroxides such as lauroyl peroxide and benzoyl peroxide.

In preferred embodiments utilizing a peroxide as the oxidizing agent, the peroxide should be of high purity and in the form of finely divided crystalline particles, preferably micronized particles having a mean average particle size of less than 35 microns. A preferred peroxide for use in the present invention is benzoyl peroxide, and the most preferred is micronized benzoyl peroxide 70% hydrous.

The oxidizing agent should be present in the composition in a pharmaceutically effective concentration. Such concentration will vary depending on the type of oxidizing agent used and the nature of the individual being treated. For guideline purposes, it is recommended that the composition contain about 1% to about 30% w/w-composition of oxidizing agent and preferably about 2.5% to about 15% w/w-composition oxidizing agent. In especially preferred embodiments, the oxidizing agent is at a concentration of about 3 to about 6% w/w-composition.

Active ingredients which are used in formulating the composition of the present invention and which are normally in solid form are advantageously converted into a liquid form in the preparation of the components from which the composition is made. For example, to prepare a liquid form of an active ingredient, it is combined with an appropriate solvent, the identity of which will depend on the particular active ingredient used. For example, if the active ingredient is highly soluble in water, then water may be used as the solvent. Water may also be added to the composition as a diluent. If the active ingredient has low solubility in water, another appropriate solvent such as an alcohol may be used as the solvent.

Any pharmaceutically acceptable and suitable material can be used to impart to the component the desired viscosity. Viscosity-enhancing agents are well known. Preferably, a gelling agent is used. The gelling agent may be selected both as to type and quantity to give products of desired viscosities.

A variety of gelling agents may be used in the practice of the invention. Preferred gelling agents are pure micro-crystalline cellulose, colloidal magnesium-aluminum silicate, hydroxypropyl methyl cellulose and the so-called hydroxylated vinylic polymers, particularly, those disclosed in U.S. Pat. No. 2,798,053. Preferred hydroxylated vinylic polymers are those described generally as interpolymers of a monomeric monolefinic acrylic acid and from about 0.1% to about 10% by weight based on the total monomer of a monomeric polyether of an oligosaccharide in which the hydroxyl groups that are modified are esterified with allyl groups, with said polyether containing at least two allyl ether groups per oligosaccharide molecule. Commercially available interpolymers of this type are known as “carbomers” marketed under the trademark Carbopol®. Such interpolymers are described as being polymers of acrylic acid cross-linked with about 1% of a polyallyl ether of sucrose having an average of about 5.8 allyl groups for each sucrose molecule. These polymers have molecular weight in the order of magnitude of about 1,000,000. Such polymers are available from the B. F. Goodrich Chemical Company and are sold under such trademarks as Carbopol® 934, Carbopol® 940, and Carbopol® 941. A particularly preferred Carbopol is Carbopol® 934 NF.

The gelling agent should be present in the composition at a concentration that provides sufficient viscosity to the composition to allow the composition to adhere to the skin for a sufficient period of time to allow the active ingredients to act on the affected areas. The effective concentration will depend on the particular gelling agent used and the nature of the component to be gelled. In some embodiments, two or more gelling agents will be present. For guideline purposes, it is recommended that the gelling agent(s) comprise about 1 to about 4% w/w-composition, preferably about 1.5 to about 3% w/w-composition.

In a highly preferred composition of the present invention, hydroxypropylcellulose is used as a gelling agent.

In a preferred embodiment, a carbomer such as the Carbopol® interpolymers described hereinabove are used as a gelling agent in the oxidizing agent component and hydroxypropylcellulose is used as a gelling agent in the antibiotic component. Hydroxypropylcellulose is a propylene glycol ether of cellulose.

The preferred hydroxypropylcellulose used in the practice of the invention is available from Aqualon as Klucel® GF having a weight average molecular weight of 370,000.

A surface active agent may be included in the composition. The surface active agent functions as a processing aid which facilitates even distribution of the oxidizing agent (such as benzoyl peroxide) in the oxidizing agent component gel. The surface active agent may also function to retard the degradative interaction between an oxidizing agent and an antibiotic. Any pharmaceutically acceptable surface active agent may be used. An example of such a surface active agent is dioctyl sodium sulfosuccinate. The surface active agent is present at a concentration sufficient to facilitate even distribution of the oxidizing agent and optionally to retard the aforementioned degradative interaction. For guideline purposes, it is recommended that the surface active agent comprise about 0.05% to about 1% w/w-composition. In preferred embodiments, dioctyl sodium sulfosuccinate 75% is present at a concentration of about 0.07 to about 0.3% w/w-composition.

The preferred composition includes also a base. The base is added to neutralize the gelling agent (carbomer). The preferred pH range for the carbomer containing gels is about 3 to about 7. For guideline purposes, the concentration is about 0.1 to about 1% w/w-composition, preferably about 0.15 to about 0.4% w/w-composition. Suitable bases include, but are not limited to, sodium hydroxide. Other bases, including amines and hydroxides, may be used, for example, triethanolamine, PEG-15 cocamine and potassium hydroxide. In especially preferred embodiments, sodium hydroxide is used at a concentration of about 0.2 to about 0.35% w/w-composition.

Examples of other optional ingredients that may be present in an acne-treating composition are as follows. The composition can include a moistening agent (for example, propylene glycol) at a concentration that functions as a solvent for methyl- and propylparabens. Preferably the moistening agent comprises about 0.5 to about 4% w/w-composition, most preferably about 1.5 to about 2.5% w/w-composition. The composition may include also an anti-microbial preservative (for example, methylparaben and propylparaben) at a concentration sufficient to inhibit microbial growth in the composition. Preferably, the composition comprises about 0.01 to about 1% w/w-composition, most preferably about 0.03 to about 0.07% w/w-composition.

As mentioned above, a preferred composition for use in the practice of the present invention includes pharmaceutically effective amounts of benzoyl peroxide and erythromycin and hydroxypropylcellulose. A particularly preferred form of this composition comprises: (A) benzoyl peroxide at a concentration of about 2.5 to about 15% w/w-composition, most preferably about 3 to about 6% w/w-composition; (B) erythromycin at a concentration of about 0.8 to about 5% w/w-composition, most preferably about 1.8 to about 4.5% w/w-composition; and (C) hydroxypropylcellulose at a concentration of about 0.5 to about 4% w/w-composition, most preferably about 1 to about 3% w/w-composition.

Preferably the composition includes an additional gelling agent, preferably Carbopol® 934 at a concentration of about 0.5 to about 2% w/w-composition, most preferably about 0.7 to about 1.5% w/w-composition. In this particularly preferred form of the composition, water is used to dissolve the benzoyl peroxide at a concentration of about 35 to about 45% w/w-composition, most preferably about 42 to about 44% w/w-composition and an alcohol, preferably ethanol (SD #40B Alcohol as described in 27 CFR §21.76) is used to dissolve the erythromycin at a concentration of about 40 to about 48% w/w-composition, most preferably about 43 to about 45% w/w-composition. Optional, but preferred ingredients, of this particularly preferred form of the composition comprise sodium dioctyl sulfosuccinate 75% at a concentration of about 0.05 to about 0.5% w/w-composition, most preferably about 0.08 to about 0.12% w/w-composition and sodium hydroxide at a concentration of about 0.05 to about 0.4% w/w-composition, most preferably about 0.1 to about 0.3% w/w-composition.

There follows a description of exemplary components which can be combined to form the acne-treating composition of the present invention.

Compositions for use in the practice of the present invention are prepared by combining at least two separate active ingredient-containing components. Preferably, a first component used to prepare the composition contains an oxidizing agent and the second component contains an antibiotic. In preferred embodiments of the acne-treating composition, the oxidizing agent is present in equal or greater amounts than the antibiotic, preferably the ratio of oxidizing agent to antibiotic being about 1:1 to about 30:1. In highly preferred embodiments, the ratio of oxidizing agent to antibiotic is about 1:1 to about 5:1.

In preferred embodiments of the present invention, the antibiotic component comprises clindamycin or erythromycin, a solvent for the antibiotic, and a gelling agent. Additional ingredients which may be present include moistening agents, anti-microbial agents and a base.

In a preferred antibiotic component containing clindamycin (“clindamycin component”), the clindamycin is in the form of clindamycin phosphate and is present in a concentration such that the acne-treating composition includes a pharmaceutically effective concentration of the clindamycin phosphate. A milligram of clindamycin phosphate contains approximately 841 μg of clindamycin. Accordingly, a 0.7-2% w/w-composition clindamycin will require approximately 0.8-2.3% w/w-composition clindamycin phosphate. Preferably the clindamycin phosphate concentration comprises about 1.5 to about 3.5% w/w-component. The clindamycin component includes additionally a solvent for the clindamycin. In preferred embodiments, the solvent is purified water which comprises about 88 to about 93% w/w-component. The clindamycin component also includes a gelling agent which imparts thereto a viscosity (about 300,000 to about 500,000 cp) which is similar to the viscosity of the other component used in the composition (preferably a benzoyl peroxide-containing component). The preferred gelling agent is Carbopol® 934 which comprises about 1 to about 2% w/w-component. The clindamycin component can also include a base for the purpose of neutralizing the carbomer (Carbopol® 934) gelling agent. The desired pH range for the carbomer gels is about 3 to about 7. Preferably the base is sodium hydroxide in a concentration of about 0.1 to about 0.3 w/w-component. Other preferred ingredients in the clindamycin component are: (A) the moistening agent prqpylene glycol at a concentration of about 2 to about 6% w/w-component; and (B) the antimicrobial agents methylparaben and propylparaben at concentrations of about 0.04 to about 0.2% w/w-component.

In a preferred antibiotic component containing erythromycin, the erythromycin is present in a concentration such that the acne-treating composition includes a pharmaceutically effective concentration of the erythromycin. Preferably the erythromycin concentration comprises about 5.5 to about 8.5% w/w-component. As erythromycin has limited solubility in water, the erythromycin is dissolved in alcohol, preferably SD alcohol #40-B 190 proof, in a concentration of about 87 to about 92% w/w-component. The erythromycin component also includes a gelling agent which imparts to the component a viscosity (about 200,000 cps to about 400,000 cps) that is similar to the viscosity of the other component used in the composition (preferably a benzoyl peroxide-containing component). Hydroxypropylcellulose is the preferred gelling agent and is present preferably in a concentration of about 3 to about 5% w/w-component.

A preferred oxidizing agent component comprises benzoyl peroxide (for example, 70% hydrous) in a concentration such that the acne-treating composition contains a pharmaceutically effective concentration of the benzoyl peroxide. Preferably the benzoyl peroxide comprises about 8 to about 12% w/w-component. The benzoyl peroxide. is preferably dissolved in water at a concentration of about 80 to about 85% w/w-component. The oxidizing agent component also includes a gelling agent which imparts to the component a viscosity (about 200,000 to about 500,000 cp) which is similar to the viscosity of the other component used to prepare the acne-treating composition (preferably an antibiotic component). A preferred gelling agent is Carbopol® 934 at a concentration of about 1 to about 3% w/w-component. The preferred oxidizing agent component comprises also the surface active agent dioctyl sodium sulfosuccinate at a concentration of about 0.1 to 0.3% w/w-component. Another preferred ingredient of the oxidizing agent component is sodium hydroxide which is present at a concentration of about 0.3 to 0.5% w/w-component.

### EXAMPLES

The following examples are illustrative of the present invention. The term “USP” as used herein refers to chemicals that conform to the tolerances set forth in the United States Pharmacopeia/National Formulary. (The term “NF” refers to National Formulary standards.) The United States Pharmacopeia/National Formulary is a compendium of pharmaceutical formulations widely used as a standard reference. The term “USP” refers to United States Pharmacopeia standards. The USP/NF is maintained by the United States Pharmacopeial Convention, Inc. The USP24/NF19 was issued January, 2000.

### Example 1

Preparation of Benzoyl Peroxide Gel/Erythromycin Gel Package

(A) Benzoyl Peroxide Gel

A benzoyl peroxide gel containing the following ingredients was prepared as follows.

| Ingredients | Weight Percent |
|---|---|
| Purified Water, USP | 82.495 |
| Carbopol ® 934, NF | 1.900 |
| Sodium Hydroxide, NF | 0.405 |
| Benzoyl Peroxide, USP 70% | 15.000 |
| Dioctyl Sodium Sulfosuccinate 75% | 0.200 |

Purified water was added to a tank (hereinafter, the “Phase A tank”) and sodium hydroxide pellets were added to the water. The contents of the tank were mixed until the pellets fully dissolved in the water and the solution cooled to 22-30° C.

Purified water was introduced into a second tank (hereinafter, the “main tank”) and the water was cooled to 22-25° C. Carbopol® 934 was dispensed into the main tank and the tank mixed. The contents of the Phase A tank were slowly added to the contents of the main tank while continuously mixing the contents of the main tank at a temperature of 22-30° C.

Purified water was added to a third tank (hereinafter, the “phase B tank”). The dioctyl sodium sulfosuccinate was added to the tank, followed by addition of benzoyl peroxide. The contents of the tank were mixed producing a slurry. The Phase B tank was connected to the main tank and the benzoyl peroxide slurry was pumped into the main tank.

The contents of the main tank were mixed under a 10-15″ psi vacuum and transferred to clean and sanitized bulk storage containers. The resultant gel had a viscosity of 360,000 cps. The viscosity was tested using method 1V-101 Brookfield helipath T-F @ 1.5 rpm.

(B) Erythromycin Gel

An erythromycin gel containing the following ingredients was prepared as follows.

| Ingredients | Weight Percent |
|---|---|
| S.D. Alcohol #40-B 190 Proof | 89.10 |
| Erythromycin, USP | 6.90 |
| Hydroxypropylcellulose, NF | 4.00 |

Specially denatured (SD) Alcohol #40-B 190 proof was added to a vessel. Erythromycin was added while mixing. Mixing was continued until the erythromycin completely dissolved. Hydroxypropylcellulose (Klucel® GF manufactured by Aqualon) was then added while mixing. Mixing was performed at −15 to −30 psi to -facilitate deaeration. Mixing may be performed in an open or closed vessel. For commercial preparations, mixing will be performed in a closed vessel. After mixing, the tank was sealed and held for a minimum of 12 hours at room temperature in order to allow any entrapped air to dissipate and to assure that all of the hydroxypropylcellulose had gelled. Any evaporated alcohol was replaced with SD Alcohol #40-B to the target weight and the mixture was mixed at −15 to −30 psi. Mixing may be performed in an open or closed vessel. For commercial preparations, mixing will be performed in a closed vessel. The mixture was then transferred into clean and sanitized steel drums. The resultant gel had a viscosity of 360,000 cps. The viscosity was tested using method 1V-101 Brookfield helipath T-F @ 1.5 rpm.

(C) Packaging the Gels

The packaging is performed using a Bartelt horizontal, form-fill-and-seal machine.

The benzoyl peroxide and the erythromycin gels were transferred to pressure vessels charged by inert gas. A pressure regulator was used to keep the pressure level constant. The pressure level was set up to 80 psi for benzoyl peroxide and up to 40 psi for erythromycin gel. A vertical linear piston-metering chamber was installed in each of the gel filling lines. The volume of gel in the chamber was kept constant by the pressurized vessels continuously forcing gel into the metering chamber which assures filling precision and accuracy of viscous gels at high speeds and low volumes. The stroke of the piston in the metering chamber is initiated by a programmable logic controller (PLC) signal synchronized with the movement of the rest of the filling line. The piston drives the gels to a filling pump. A horizontal linear positive displacement micro-pump (Hi-Bar pump), with a packet-dosing chamber, was installed at the terminal end of the filling line. The dosing chamber was filled by the action of the metering chamber piston. The dosing chamber feeds a loop of clear tubing terminating in a vertical piston, at the filling station.

The gels are packaged in a two-packet laminated foil package containing the benzoyl peroxide gel in one packet and the erythromycin gel in the other packet. The benzoyl peroxide gel (0.425 g) was filled into one packet while the erythromycin gel (0.425 g) was simultaneously filled into the second packet. The dispensed gels are within plus or minus 0.025 g of the target weight of 0.425 g, allowing for a 6% tolerance. After each packet was filled and sealed, the packets were folded and cold-sealed together using a glue adhesive. The packets are constructed of a laminated foil consisting of an inner barrier of low-density polyethylene; a tie layer of ethylene/acrylic acid copolymer; a main barrier of foil; a tie layer of ethylene/acrylic acid copolymer; and an outer barrier of polyester.

Combining the benzoyl peroxide gel with the erythromycin gel provides an acne-treating composition having the following ingredients by weight %.

| | % W/W |
|---|---|
| INGREDIENT | COMPOSITION |
| Purified Water, USP | 43.50 |
| Carbopol ® 934, NF | 0.95 |
| Sodium Hydroxide, NF | 0.20 |
| Benzoyl Peroxide, 70% Hydrous, USP | 5.25\hat{ } |
| Dioctyl Sodium Sulfosuccinate, DF | 0.10 |
| SD Alcohol #40-B/190°, DF | 44.55 |
| Erythromycin, USP | 3.45 |
| Hydroxypropylcellulose, NF | 2.00 |

### Example 2

Preparation of Benzoyl Peroxide/Clindamycin Gel Package

The benzoyl peroxide gel is prepared as described in Example 1.

A clindamycin gel containing the following ingredients was prepared as follows.

| Ingredients | Weight Percent |
|---|---|
| Purified Water, USP | 91.28 |
| Carbomer 934 | 1.50 |
| Sodium Hydroxide, NF | 0.27 |
| Propylene Glycol, USP | 4.00 |
| Methylparaben, NF | 0.14 |
| Propylparaben, NF | 0.06 |
| Clindamycin Phosphate, USP | 2.75 |

Purified water was added to a tank (hereinafter, the “Phase A tank”) and sodium hydroxide pellets were added to the purified water and the contents of the tank were mixed until the pellets fully dissolved in the purified water and the solution cooled to 22-30° C.

Purified water was added into a second tank (hereinafter, the “main tank”) and Carbomer 934 was dispensed into the main tank. The contents of the phase A tank were slowly added to the contents of the main tank while continuously mixing the contents of the main tank and deaerating under a vacuum. The temperature was maintained at 22-30° C.

Purified water was added to a third tank (hereinafter, the “phase B tank”). Clindamycin phosphate was added and mixed until dissolved. Once the clindamycin phosphate was completely dissolved, the contents of the tank were pumped into the main tank and mixed under vacuum.

Purified water and propylene glycol were added to a fourth tank. The tank was heated to 35-40° C. Methylparaben was then added and the contents of the tank were mixed until all the methylparaben was dissolved. Propylparaben was then added and the contents of the tank mixed until all the propylparaben was dissolved. The contents were transferred into the main tank.

While continuously mixing, a vacuum was turned on and the mixing continued until the material was homogenous. The contents of the tank were then transferred to stainless steel drums. The resultant gel had a viscosity of 370,000 cps. The viscosity was tested using method IV-101 Brookfield helipath T-P @ 1.5 rpm.

The benzoyl peroxide and the erythromycin gels were transferred to pressure vessels charged by inert gas. A pressure regulator was used to keep the pressure level constant. The pressure level was set up to 80 psi for benzoyl peroxide and up to 40 psi for erythromycin gel. A vertical linear piston-metering chamber was installed in each of the gel filling lines. The volume of gel in the chamber was kept constant by the pressurized vessels continuously forcing gel into the metering chamber which assures filling precision and accuracy of viscous gels at high speeds and low volumes. The stroke of the piston in the metering chamber is initiated by a PLC signal synchronized with the movement of the rest of the filling line. The piston drives the gels to a filling pump. A horizontal linear positive-displacement micro-pump, with a packet dosing chamber, was installed at the terminal end of the filling line. The dosing chamber was filled by the action of the metering chamber piston. The dosing chamber feed a loop of clear tubing terminating in a vertical piston, at the filling station.

For the final packaging step, 0.425 g of a 10% benzoyl peroxide gel was filled into one packet while 0.425 g of a 2% clindamycin gel was simultaneously filled into the second packet. The dispensed gels are within plus or minus 0.025 g of the target weight of 0.425 g, allowing for a 6% tolerance. After each pouch was filled and sealed, the pouches were folded and cold-sealed together.

Combining the benzoyl peroxide gel with the clindamycin gel provides an acne-treating composition having the following ingredients by weight %.

| INGREDIENT | % W/W COMPOSITION |
|---|---|
| Purified Water, USP | 89.14 |
| Carbomer 934, NF | 1.70 |
| Sodium Hydroxide, NF | 0.34 |
| Propylene Glycol, USP | 2.00 |
| Dioctyl Sodium Sulfosuccinate 75%, DF | 0.10 |
| Methylparaben, NF | 0.07 |
| Propylparaben, NF | 0.03 |
| *Clindamycin Phosphate, USP | 1.37 |
| Hydrous Benzoyl Peroxide, USP | 5.25 |

*The % w/w for the clindamycin phosphate is based solely on % w/w. The active substance in the product is clindamycin. Clindamycin phosphate is converted in vitro to active clindamycin. The actual amount of clindamycin as clindamycin phosphate is based on several factors including activity. The actual amount of material added is based on 6.875 kg of clindamycin phosphate.

1. A package comprising components which, upon being mixed, are capable of forming a pharmaceutical composition that is effective in treating acne, said composition tending to degrade prematurely, one of said components comprising an oxidizing agent and another of said components comprising an antibiotic which is effective against acne-associated bacterial species, said components separated one from the other in said package, said one component having a viscosity within about 50% of the viscosity of said other component.

2. The package of claim 1 wherein said component comprising an oxidizing agent comprises about 8 to about 12% w/w-component oxidizing agent.

3. The package of claim 1 wherein said component comprising an antibiotic comprises about 1.5 to about 8.5% w/w-component antibiotic.

4. The package of claim 1 wherein the ratio of said oxidizing agent to said antibiotic is about 1:1 to about 30:1.

5. The package of claim 4 wherein the ratio of said oxidizing agent to said antibiotic is about 1:1 to about 5:1.

6. The package of claim 1 wherein said oxidizing agent is benzoyl peroxide.

7. The package of claim 1 wherein said antibiotic is selected from the group consisting of erythromycin and clindamycin.

8. The package of claim 7 wherein said antibiotic is erythromycin.

9. The package of claim 1 wherein said package comprises a dual packet laminated foil package.

10. A package comprising components which, upon being mixed, are capable of forming a pharmaceutical composition that is effective in treating acne, one of said components comprising a benzoyl peroxide gel and another of said components comprising a gel of erythromycin, said components separated one from the other, said benzoyl peroxide gel having a viscosity within about 50% of the viscosity of said erythromycin gel.

11. The package of claim 10 wherein said benzoyl peroxide gel comprises about 8 to about 12% w/w-component benzoyl peroxide.

12. The package of claim 11 wherein said benzoyl peroxide gel comprises about 10% w/w-component benzoyl peroxide.

13. The package of claim 10 wherein said erythromycin gel comprises about 5.5 to about 8.5% w/w-component erythromycin.

14. The package of claim 13 wherein said erythromycin gel comprises about 7% w/w-component erythromycin.

15. The package of claim 14 wherein the ratio of said benzoyl peroxide to said erythromycin is about 1:1 to about 30:1.

16. The package of claim 15 wherein the ratio of said benzoyl peroxide to said erythromycin is about 1:1 to about 5:1.

17. The package of claim 10 wherein said package is a dual packet laminated foil package.

18. A package comprising: (A) components which, upon being mixed, are capable of forming a pharmaceutical composition that is effective in treating acne, one of said components comprising a benzoyl peroxide gel and another of said components comprising a gel of erythromycin and hydroxypropylcellulose; and (B) containers for holding said components in the package separated one from the other; said components having viscosities such that, upon the application of a uniform force to said components, substantially equal volumes of said components are capable of being dispensed simultaneously from said containers.

19. The package of claim 18 wherein said benzoyl peroxide gel comprises about 8 to about 12% w/w-component benzoyl peroxide.

20. The package of claim 18 wherein said erythromycin and hydroxypropylcellulose gel comprises about 5.5 to about 8.5% w/w-component erythromycin and about 3 to about 5% w/w-component hydroxypropylcellulose.

21. The package of claim 18 wherein said benzoyl peroxide gel comprises about 10.5% w/w-component benzoyl peroxide and said gel of erythromycin and hydroxypropylcellulose comprises about 7% w/w-component erythromycin and about 4% w/w-component hydroxypropylcellulose.

22. The package of claim 21 wherein said benzoyl peroxide gel has a viscosity from about 200,000 cps to about 500,000 cps and said erythromycin and hydroxypropylcellulose gel has a viscosity of from about 200,000 cps to about 400,000 cps.

23. A composition for the treatment of acne comprising:
(a) benzoyl peroxide;
(b) erythromycin; and
(c) hydroxypropylcellulose.

24. The composition of claim 23 including an additional gelling agent.

25. The composition of claim 23 including a surface active agent.

26. The composition of claim 23 including a base which increases the pH of said composition to a value at which said composition has an acceptable viscosity.

27. The composition of claim 23 including an alcohol solvent for said erythromycin.

28. The composition of claim 23 including a diluent.

29. A composition for the treatment of acne comprising:
(a) benzoyl peroxide; (b) erythromycin; (c) hydroxypropylcellulose; (d) a gelling agent; (e) a surface active agent; (f) a base; (g) an alcohol; and (h) water.

30. The composition of claim 29 wherein said gelling agent is a hydroxylated vinylic polymer.

31. The composition of claim 29 wherein said surface active agent is dioctyl sodium sulfosuccinate.

32. The composition of claim 29 wherein said base is sodium hydroxide.

33. The composition of claim 29 wherein said alcohol is specially denatured ethanol.

34. A composition for the treatment of acne comprising about 5.25% w/w-composition benzoyl peroxide, about 3.45% w/w-composition erythromycin, and about 2% w/w-composition hydroxypropylcellulose.

35. The composition of claim 34 comprising about 0.95% w/w-composition of a hydroxylated vinylic polymer, about 0.10% w/w-composition of dioctyl sodium sulfosuccinate, about 0.2% w/w-composition of sodium hydroxide, about 44.55% w/w-composition specially denatured ethanol and about 43.5% w/w-composition of water.

36. A package containing a first acne-treating composition and a second acne-treating composition, said compositions tending to degrade chemically upon being mixed with one another, said package comprising a first chamber containing said first composition and a second chamber containing said second composition, each of said chambers having a sealed dispensing orifice, said orifices positioned adjacent to one another.

37. A package containing a first acne-treating composition and a second acne-treating composition, said compositions tending to degrade chemically upon being mixed with one another, said package comprising a first and second packet, said packets being attached to each other and each of said packets having a sealed dispensing orifice, said sealed dispensing orifices being positioned adjacent to each other so that, as said compositions are dispensed through the orifices in unsealed form, they are combined with each other.

38. A package containing a first acne-treating composition and a second acne-treating composition, said compositions tending to degrade chemically upon being mixed with one another, said package comprising a first and second packet, each of said packets having a top which includes a dispensing orifice that has a tear-off tab seal, a bottom, two sides in a parallel relationship to one another, and an exterior face and an interior face, said first packet containing said first composition and said second packet containing said second composition, said interior faces of said packets adjacent to and facing one another such that said tear-off tabs can be simultaneously torn off such that said compositions are dispensed and combined with each other as they exit said packets through said dispensing orifices.
